(19) United States
(12) Patent Application Publication
(10) Pub. No.: US 2012/0036768 A1
(43) Pub. Date: Feb. 16, 2012

(54) HIGH CONSISTENCY ENZYMATIC HYDROLYSIS FOR THE PRODUCTION OF ETHANOL

(76) Inventors: Richard Phillips, Raleigh, NC (US); Hasan Jameel, Cary, NC (US); Hou-Min Chang, Raleigh, NC (US)

(21) Appl. No.: 13/130,521

(22) PCT Filed: Nov. 23, 2009
(86) PCT No.: PCT/US09/65562

§ 371 Date: Sep. 16, 2011

Related U.S. Application Data
(60) Provisional application No. 61/116,909, filed on Nov. 21, 2008.

(51) Int. Cl. C10L 1/182; C12P 7/14; C07C 31/08; C12P 7/02

(52) U.S. Cl. 44/451; 435/155; 435/162; 568/840

(57) ABSTRACT

The presently disclosed subject matter related to methods of converting lignocellulosic materials to alcohol that include increasing the fiber consistency of enzymatic hydrolysis mixtures. More particularly, the methods involve contacting lignocellulosic biomass with an enzyme composition for a period of time, and then thickening the mixture and further hydrolyzing the thickened mixture. The thickening can be performed by filtration, optionally reusing the filtrate and/or any enzymes contained therein during the lignocellulose conversion process to increase the efficiency of the process. Hydrolysis of the thickened mixture provides a fermentable sugar mixture having a higher concentration of sugar than fermentable sugar mixtures provided from less concentrated biomass/enzyme mixtures. Compositions comprising alcohol prepared according to the presently disclosed methods are also provided.

### RELATED APPLICATIONS

This application claims the benefit of U.S. Provisional Patent Application No. 61/116,909, filed Nov. 21, 2008, the disclosure of which is incorporated herein by reference in its entirety.

### TECHNICAL FIELD

The presently disclosed subject matter relates to methods of converting lignocellulosic biomass to alcohol employing enzymatic hydrolysis reactions performed at high fiber consistency. The use of high fiber consistency enzymatic hydrolysis reactions allows for the recovery of high sugar concentration mixtures that subsequently can provide high alcohol content solutions following fermentation. Also provided are methods of recycling lignocellulose-hydrolysis enzymes.

### ABBREVIATIONS

° C.=degrees Celsius
%=percentage
% K=fiber concentration or fiber consistency
b=β-glucosidase
c=cellulase
EH=enzymatic hydrolysis
FPU=filter paper units
GL=green liquor
gm=gram
hr=hours
HW=hardwood
L=liters
min=minutes
Na₂CO₃=sodium carbonate
NaOH=sodium hydroxide
Na₂S=sodium sulfide
Na₂SO₄=sodium sulfate
SSF=simultaneous saccharification and fermentation
TTA=total titratable alkali
x=xylanase

### BACKGROUND

Plant-derived lignocellulosic biomass represents a large, renewable source of potential starting materials for the production of a variety of chemicals, plastics, fuels and feeds. For example, lignocellulosic biomass feedstocks comprise cellulose, a polymer of glucose, which can be hydrolyzed to provide fermentable sugar for use in the production of ethanol.

Cellulose hydrolysis can be performed by acid or enzymatic hydrolysis (EH). In EH, a family of enzymes can be used that works together to hydrolyze glycosidic bonds in polymeric lignocellulose molecules. Most EH is done at a lignocellulose fiber concentration (which can be referred to as a % K), between about 5-10%, to ensure proper contact between the enzymes and the fibers. At higher fiber concentrations, the cellulose can swell to provide very thick mixtures that are hard to handle (e.g., transfer from one reactor to another) and/or that make proper mixing of the enzymes and the fibers difficult, thus reducing hydrolysis efficiency. Unfortunately, the low concentration of fibers during hydrolysis results in solutions containing low concentrations of simple sugars, increasing the size of fermentation vessels that must be used during the ethanol production processes. Low sugar concentration also leads to lower alcohol concentration following fermentation, requiring larger distillation columns and higher energy input for purification of fermented mixtures.

Accordingly, there is a need for efficient methods of hydrolyzing lignocellulosic materials at higher fiber concentrations (e.g., >10% K). Such methods can be used to provide higher concentration glucose solutions, resulting in significant capitol and operating savings in alcohol production plants.

### SUMMARY

The presently disclosed subject matter provides, in some embodiments, a method of producing an alcohol from a lignocellulosic biomass, the method comprising: providing lignocellulosic biomass; contacting the lignocellulosic biomass with a first enzyme composition for a first period of time to provide a first hydrolysis mixture; thickening the first hydrolysis mixture to form a second hydrolysis mixture; hydrolyzing the second hydrolysis mixture for a second period of time to provide a fermentable sugar mixture; and fermenting the fermentable sugar mixture to provide an alcohol.

In some embodiments, the lignocellulosic biomass is selected from the group consisting of herbaceous material, agricultural residues, forestry residues, municipal solid wastes, waste paper, pulp and paper mill residues, or a combination thereof. In some embodiments, the lignocellulosic biomass is selected from the group consisting of corn stover, straw, bagasse, miscanthus, sorghum residue, switch grass, bamboo, water hyacinth, hardwood, hardwood chips, softwood chips, hardwood pulp, and softwood pulp.

In some embodiments, the method further comprises pretreating the lignocellulosic biomass to increase enzymatic digestability. In some embodiments, the pretreating comprises one or more of the group consisting of removing or altering lignin, removing hemicellulose, decrystallizing cellulose, removing acetyl groups from hemicellulose, reducing the degree of polymerization of cellulose, increasing the pore volume of lignocellulose biomass, and increasing the surface area of lignocellulose.

In some embodiments, the pretreating comprises one or more pretreatment technique selected from the group consisting of autohydrolysis, steam explosion, grinding, chopping, ball milling, compression milling, radiation, flow-through liquid hot water treatment, dilute acid treatment, concentrated acid treatment, peracetic acid treatment, supercritial carbon dioxide treatment, alkali treatment, organic solvent treatment, cellulose solvent treatment, and treatment with an aerobic fungi. In some embodiments, the alkali treatment is selected from the group consisting of sodium hydroxide treatment, lime treatment, wet oxidation, ammonia treatment, and oxidative alkali treatment. In some embodiments, the alkali treatment is green liquor treatment.

In some embodiments, contacting the lignocellulosic biomass with the first enzyme composition comprises mixing the lignocellulosic biomass with the first enzyme composition at a solids concentration of about 5%. In some embodiments, the first hydrolysis mixture comprises between about 5 filter paper units (FPU) and about 85 FPU of lignocellulose-hydrolyzing enzyme per gram of lignocellulosic biomass. In some embodiments, the first hydrolysis mixture comprises about 10 FPU of lignocellulose-hydrolyzing enzyme per gram of lignocellulosic biomass.

In some embodiments, the first enzyme composition comprises cellulase. In some embodiments, the first enzyme composition further comprises xylanase and β-glucosidase.

In some embodiments, the first period of time ranges from about 1 minute to about 20 minutes. In some embodiments, the first period of time ranges from about 5 minutes to about 10 minutes.

In some embodiments, contacting the lignocellulosic biomass with the first enzyme composition is performed at a temperature of between about 4° C. and about 70° C. In some embodiments, the contacting is performed at a temperature of about 38° C. In some embodiments, the contacting is performed at a pH of about 4.8.

In some embodiments, the thickening step comprises increasing the fiber concentration of the first hydrolysis mixture to provide a second hydrolysis mixture having a solids concentration of between about 15% and about 30%. In some embodiments, the thickening step comprises filtering the first hydrolysis mixture to provide the second hydrolysis mixture and a filtrate. In some embodiments, the filtering is performed by vacuum filtering the first hydrolysis mixture using a filter press.

In some embodiments, the filtrate comprises about 80% of the liquid from the first hydrolysis mixture. In some embodiments, the filtrate comprises water and unabsorbed lignocellulose-hydrolyzing enzyme, and wherein said filtrate is used to dilute lignocellulosic biomass, thereby recycling the lignocellulose-hydrolyzing enzyme. In some embodiments, the first enzyme composition comprises cellulase and wherein the filtrate comprises about 10% to about 20% of the cellulase from the first hydrolysis mixture.

In some embodiments, the second period of time ranges between about 1 day and about 3 days. In some embodiments, hydrolyzing the second hydrolysis mixture comprises hydrolyzing the second hydrolysis mixture for a first portion of the second period of time, adding a second enzyme composition to the second hydrolysis mixture to increase the enzyme dosage in the second hydrolysis mixture, and continuing hydrolysis of the second hydrolysis mixture for a second portion of the second period of time to provide the fermentable sugar mixture. In some embodiments, the first portion of the second period of time ranges between about 0 hours and about 24 hours. In some embodiments, the first portion of the second period of time ranges between about 2 hours and about 3 hours.

In some embodiments, the second enzyme composition comprises xylanase and β-glucosidase. In some embodiments, the second enzyme composition further comprises cellulase.

In some embodiments, the first enzyme composition and the second enzyme composition each comprise cellulase, and the first enzyme composition comprises between about 25% and about 50% of the total cellulase dosage from the first and second enzyme compositions. In some embodiments, the first enzyme composition comprises about 50% of the total cellulase dosage.

In some embodiments, the second portion of the first period of time ranges between about 24 hours and about 48 hours.

In some embodiments, hydrolysis efficiency of cellulosic material originally present in the lignocellulosic biomass is about 70% or greater. In some embodiments, the hydrolysis efficiency is between about 78% and about 84%. In some embodiments, the fermentable sugar mixture comprises about 12% fermentable sugar by volume.

In some embodiments, fermenting comprises fermenting the fermentable sugar mixture using a microorganism to provide an alcohol mixture and distilling the alcohol mixture to provide the alcohol. In some embodiments, the microorganism is yeast. In some embodiments, the alcohol mixture comprises about 6% alcohol by volume. In some embodiments, the method further comprises dehydrating the alcohol. In some embodiments, the alcohol is ethanol.

In some embodiments, the presently disclosed subject matter provides a composition comprising an alcohol prepared according to a method comprising: providing lignocellulosic biomass; contacting the lignocellulosic biomass with a first enzyme composition for a first period of time to provide a first hydrolysis mixture; thickening the first hydrolysis mixture to from a second hydrolysis mixture; hydrolyzing the second hydrolysis mixture for a second period of time to provide a fermentable sugar mixture; and fermenting the fermentable sugar mixture to provide the alcohol.

In some embodiments, the alcohol is ethanol. In some embodiments, the composition comprises about 95% or greater ethanol by volume. In some embodiments, composition is a fuel mixture comprising ethanol and gasoline.

Accordingly, it is an object of the presently disclosed subject matter to provide methods of producing alcohol from lignocellulosic biomass wherein the method comprises enzyme hydrolysis of a high consistency lignocellulosic biomass mixture, as well as compositions comprising alcohol produced thereby.

Certain objects of the presently disclosed subject matter having been stated hereinabove, which are addressed in whole or in part by the presently disclosed subject matter, other objects and advantages will become apparent to those of ordinary skill in the art after a study of the following description of the presently disclosed subject matter and in the accompanying non-limiting Examples.

### BRIEF DESCRIPTION OF THE DRAWINGS

FIG. 1A is a block diagram showing a method for preparing a sugar mixture from lignocellulosic biomass according to an embodiment of the presently disclosed subject matter.

FIG. 1B is a block diagram showing a method for preparing alcohol from lignocellulosic biomass according to an embodiment of the presently disclosed subject matter.

FIG. 2A is a graph showing the effect of fiber consistency (% K) on the enzymatic hydrolysis (EH) of wood chips following pretreatment with green liquor (GL) having total titratable alkali of 12%. The data shown by diamonds represents EH performed at 5K. The data shown by squares represents EH performed at 7.5K. The data shown by triangles represents EH performed at 10% K. Enzymatic hydrolysis results are provided as % biomass conversion based on total sugars (glucose, xylose and mannose) in the hydrolysis mixture after EH.

FIG. 2B is a graph showing the effect of fiber consistency (% K) on the enzymatic hydrolysis (EH) of wood following pretreatment with green liquor (GL) having total titratable alkali of 16%. The data shown by diamonds represents EH performed at 5% K. The data shown by squares represents EH performed at 7.5% K. The data shown by triangles represents EH performed at 10% K. Enzymatic hydrolysis results are provided as % biomass conversion based on total sugars (glucose, xylose and mannose) in the hydrolysis mixture after EH.

FIG. 3 is a graph showing the influence of temperature on cellulase adsorption to wood pulp. Data is shown for cellulase adsorption at 4, 23, 38, and 50° C. Wood pulp was incubated with cellulase at 5 percent consistency (% K) for ten minutes at the indicated temperature and then thickened to 20% K. The amount of cellulase in the filtrate was determined and used to calculate the percentage of the cellulase adsorption (i.e., the % of the cellulase dosage remaining in the pulp mixture).

FIG. 4 is a graph showing the influence of cellulase dosage on cellulase adsorption to wood pulp. Data is shown for cellulase dosages ranging from 5 filter paper units (FPU)/gram (gm) wood fiber to 40 FPU/gm. Wood pulp was incubated with cellulase at 5 percent consistency (% K) at the indicated dosage for ten minutes and then thickened to 20% K. The amount of cellulase in the filtrate was determined and used to calculate the percentage of the cellulase adsorption (i.e., the % of the cellulase dosage remaining in the pulp mixture).

FIG. 5 is a graph showing the influence of lignin content on cellulase adsorption to wood pulp. Data is shown for wood pulp having from 0% lignin content to 28% lignin content. Wood pulp was incubated with cellulase at 5 percent consistency (% K) for ten minutes and then thickened to 20% K. The amount of cellulase in the filtrate was determined and used to calculate the percentage of the cellulase adsorption (i.e., the % of the cellulase dosage remaining in the pulp mixture).

FIG. 6 is a bar graph showing the effects of different green liquor (GL) pretreatments on enzyme adsorption to the pretreated wood pulp. GL-12 represents GL pretreatment at 12% total titratable alkali (TTA). GL-16 represents GL pretreatment at 16% TTA. The pretreated wood pulp was incubated with cellulase at 5 percent consistency (% K) for ten minutes and then thickened to 20% K. The amount of cellulase in the filtrate was determined and used to calculate the percentage of the cellulase adsorption (i.e., the % of the cellulase dosage remaining in the pulp mixture).

FIG. 7 is a graph showing the dependence of sugar recovery efficiency based on enzyme dosage after 48 hours of enzymatic hydrolysis (EH). Total enzyme dosage varied between 5 filter paper units (FPU) and 40 FPU/gram of wood pulp. The shaded diamonds show data for sugar recovery after enzymatic hydrolysis at 20% fiber consistency (% K) when an enzyme composition including cellulase (c), xylanase (x) and β-glucosidase (b) are all added at the same time (cxb). The shaded squares show data relating to sugar recovery after EH carried out at 20% K when cellulase is added first, followed by the addition of xylanase and β-glucosidase (c+xb). The lightly shaded triangles show data relating to EH carried out at 20% K where cellulase is added 24 hours prior to addition of xylanase and β-glucosidase (c+24 h+xb). For comparison, the open squares show the sugar recovery of EH carried out at 5% K.

FIG. 8 is a graph showing the effects on enzymatic hydrolysis efficiency of adding xylanase and β-glucosidase (i.e., xb) at different times (i.e., between 0 and 8 hours) following thickening. Data is shown for hydrolysis mixtures where hydrolysis continues for a further 24 hours (diamonds) or a further 48 hours (squares) following addition of the xylanase and β-glucosidase.

FIG. 9A is a graph showing the percentage (%) of overall enzymatic hydrolysis (EH) in bleached hardwood (HW) pulp (0% lignin content) at different enzyme dosages as determined based on total sugars produced during EH (10 minutes at 5% fiber consistency followed by 48 hours at 20% fiber consistency).

FIG. 9B is a graph showing the percentage (%) of overall enzymatic hydrolysis (EH) in hardwood (HW) pulp with 2% lignin content at different enzyme dosages as determined based on total sugars produced during EH (10 minutes at 5% fiber consistency followed by 48 hours at 20% fiber consistency).

FIG. 9C is a graph showing the percentage (%) of overall enzymatic hydrolysis (EH) in hardwood (HW) pulp with 10% lignin content at different enzyme dosages as determined based on total sugars produced during EH (10 minutes at 5% fiber consistency followed by 48 hours at 20% fiber consistency).

FIG. 9D is a graph showing the percentage (%) of overall enzymatic hydrolysis (EH) in hardwood (HW) pulp with 28% lignin content at different enzyme dosages as determined based on total sugars produced during EH (10 minutes at 5% fiber consistency followed by 48 hours at 20% fiber consistency).

FIG. 10 is a graph showing enzymatic hydrolysis (EH) efficiency using 10 filter paper units (FPU) of enzyme/gram (gm) wood pulp (28% lignin content) at either a 5% wood fiber concentration for 48 hours (stippled bars) or as described for FIG. 9D (10 minutes at 5% fiber consistency followed by 48 hours at 20% fiber consistency, hatched bars).

FIG. 11 is a graph showing enzymatic hydrolysis (EH) efficiency using 20 filter paper units (FPU) of enzyme/gram (gm) wood pulp (28% lignin content) at either a 5% wood fiber concentration for 48 hours (stippled bars) or as described for FIG. 9D (10 minutes at 5% fiber consistency followed by 48 hours at 20% fiber consistency, hatched bars).

FIG. 12 is a graph showing enzymatic hydrolysis (EH) efficiency using 40 filter paper units (FPU) of enzyme/gram (gm) wood pulp (28% lignin content) at either a 5% wood fiber concentration for 48 hours (stippled bars) or as described for FIG. 9D (10 minutes at 5% fiber consistency followed by 48 hours at 20% fiber consistency, hatched bars).

FIG. 13 is a graph showing enzymatic hydrolysis (EH) efficiency using 10 filter paper units (FPU) enzyme/gram (gm) wood pulp at either 5% fiber concentration (stippled bars), 20% fiber concentration with all enzymes added at once (hatched bars) or 20% fiber concentration with the enzyme dosage added in two equal portions (darkly shaded solid bars).

FIG. 14 is a graph showing enzymatic hydrolysis (EH) efficiency using 20 filter paper units (FPU) enzyme/gram (gm) wood pulp at either 5% fiber concentration (stippled bars), 20% fiber concentration with all enzymes added at once (hatched bars), 20% fiber concentration with the enzyme dosage added in two portions, where the first portion is 25% of the total enzyme dosage (darkly shaded solid bars), or 20% fiber concentration with the enzyme dosage added in two equal portions (medium shaded solid bars).

FIG. 15 is a graph showing enzymatic hydrolysis (EH) efficiency using 40 filter paper units (FPU) enzyme/gram (gm) wood pulp at either 5% fiber concentration (stippled bars), 20% fiber concentration with all enzymes added at once (hatched bars), 20% fiber concentration with the enzyme dosage added in two portions, where the first portion is 12.5% of the total enzyme dosage (darkly shaded solid bars), 20% fiber concentration with the enzyme dosage added in two portions, wherein the first portion is 25% of the total dosage (medium shaded solid bars), or 20% fiber concentration with the enzyme dosage added in two equal portions (unshaded open bars).

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

### I. DEFINITIONS

Following long-standing patent law convention, the terms “a” and “an” mean “one or more” when used in this application, including the claims. Thus, “an enzyme” can refer to a plurality (i.e., two or more) enzymes.

As used herein, the term “about” modifying any amount can refer to the variation in that amount encountered in real world conditions of producing sugars and ethanol, e.g., in the lab, pilot plant, or production facility. For example, the amounts can vary by about 5%, 1%, or 0.5%. Unless otherwise indicated, all numbers expressing quantities of percentage (%), temperature, time, pH, distance, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

The term “and/or” when used to describe two or more activities, conditions, or outcomes refers to situations wherein both of the listed conditions are included or wherein only one of the two listed conditions are included.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

The term “saccharide” refers to a carbohydrate monomer, oligomer or larger polymer. Thus, a saccharide can be a compound that includes one or more cyclized monomer unit based upon an open chain form of a compound having the chemical structure H(CHOH)ₙC(═O)(CHOH)ₘH, wherein the sum of n+m is an integer between 2 and 8. Thus, the monomer units can include trioses, tetroses, pentoses, hexoses, heptoses, nonoses, and mixtures thereof. In some embodiments, each cyclized monomer unit is based on a compound having a chemical structure wherein n+m is 4 or 5. Thus, saccharides can include monosaccharides including, but not limited to, aldohexoses, aldopentoses, ketohexoses, and ketopentoses such as arabinose, lyxose, ribose, xylose, ribulose, xylulose, allose, altrose, galactose, glucose, gulose, idose, mannose, talose, fructose, psicose, sorbose, and tagatose, and to hetero- and homopolymers thereof. Saccharides can also include disaccharides including, but not limited to sucrose, maltose, lactose, trehalose, and cellobiose, as well as hetero- and homopolymers thereof.

The term “oligosaccharide” refers to polysaccharides having a degree of polymerization of between about 2 and about 10.

The terms “fermentable sugar” and “sugar” can be used interchangeably and refer to oligosaccharides, monosaccharides and mixtures thereof that can be used as a carbon source in a fermentation process. Fermentable monosaccharides include arabinose, glyceraldehyde, dihydroxyacetone, erythrose, ribose, ribulose, xylose, glucose, galactose, mannose, fucose, fructose, sedoheptulose, neuraminic acid, or mixtures of these. Fermentable disaccharides include sucrose, lactose, maltose, gentiobiose, or mixtures thereof.

The term “lignocellulosic” refers to a composition comprising both lignin and cellulose. In some embodiments, lignocellulosic material can comprise hemicellulose, a polysaccharide which can comprise saccharide monomers other than glucose. Typically, lignocellulosic materials comprise between about 38-50% cellulose, 15-30% lignin, and 23-32% hemicellulose.

Lignocellulosic biomass include a variety of plants and plant materials, such as, but not limited to, papermaking sludge; wood, and wood-related materials, e.g., saw dust, or particle board, leaves, or trees, such as poplar trees; grasses, such as switchgrass and sudangrass; grass clippings; rice hulls; bagasse (e.g., sugar cane bagasse), jute; hemp; flax; bamboo; sisal; abaca; hays; straws; corn cobs; corn stover; whole plant corn, and coconut hair. In some embodiments, lignocellulosic biomass is selected from the group including, but not limited to, herbaceous material, agricultural residues, forestry residues, municipal solid wastes, waste paper, pulp and paper mill residues, or a combination thereof. In some embodiments, lignocellulosic biomass is selected from the group including, but not limited to, corn stover, straw, bagasse, miscanthus, sorghum residue, switch grass, bamboo, water hyacinth, hardwood, hardwood, softwood, wood chips, and wood pulp.

“Lignin” is a polyphenolic material comprised of phenyl propane units linked by ether and carbon-carbon bonds. Lignins can be highly branched and can also be crosslinked. Lignins can have significant structural variation that depends, at least in part, on the plant source involved.

The term “glucan” refers to a polysaccharide comprising glucose monomers linked by glycosidic bonds.

The term “cellulose” refers to a polysaccharide of β-glucose (i.e., β-1,4-glucan) comprising β-(1-4) glycosidic bonds. The term “cellulosic” refers to a composition comprising cellulose.

The term “hemicellulose” can refer polysaccharides comprising mainly sugars or combinations of sugars other than glucose (e.g., xylose). Thus, xylan (polymerized xylose) and mannan (polymerized mannose) are exemplary hemicelluloses. Hemicellulose can be highly branched. Hemicellulose can be chemically bonded to lignin and can further be randomly acetylated, which can reduce enzymatic hydrolysis of the glycosidic bonds in hemicellulose.

The terms “glycosidic bond” and “glycosidic linkage” refer to a linkage between the hemiacetal group of one saccharide unit and the hydroxyl group of another saccharide unit.

The term “biofuel” refers to a fuel that is derived from biomass, i.e., a living or recently living biological organism, such as a plant or an animal waste. Biofuels include, but are not limited to, biodisel, biohydrogen, biogas, biomass-derived dimethylfuran (DMF), and the like. In particular, the term “biofuel” can be used to refer to biomass-derived alcohols (e.g., bioalcohol), such as ethanol, methanol, propanol, or butanol, which can be denatured, if desired prior to use. The term “biofuel” can also be used to refer to fuel mixtures comprising biomass-derived fuels, such as alcohol/gasoline mixtures (i.e., gasohols). Gasohols can comprise any desired percentage of biomass-derived alcohol (i.e., about 10%, 15%, 20%, 25%, 30%, 35%, 40%, 45%, 50%, 55%, 60%, 65%, 70%, 75%, 80%, 85%, 90%, or 95% biomass-derived alcohol). For example, one useful biofuel-based mixture is E85, which comprises 85% ethanol and 15% gasoline.

The term “pretreat” generally refers to a chemical, microbial, or mechanical method of treating biomass to make it more amendable to enzymatic hydrolysis and/or microbial fermentation. For example, pretreating can relate to removing or altering lignin, removing hemicellulose, decrystallizing cellulose, removing acetyl groups (e.g., through chemical or enzymatic hydrolysis of the acetyl ester), reducing the degree of polymerization of cellulose (i.e., hydrolysis of glycosidic bonds), expanding the structure of the lignocellulosic material to increase pore volume and internal surface area.

The term “green liquor” refers to an alkaline composition, such as that used in alkaline pulping during paper production, comprising sodium sulfide (Na₂S) and sodium carbonate (Na₂CO₃). In some embodiments, the green liquor can further comprise sodium sulfate (Na₂SO₄).

The term “total titratable alkali” refers to the weight percentage of combined alkali species (e.g., Na₂CO₃, Na₂S, and NaOH) in a solution (e.g., a green liquor solution), expressed as Na₂O.

The term “sulfidity” refers to the weight percentage of alkaline sulfur compounds in a solution (e.g., a green liquor solution) compared to the total titratable alkali.

The term “delignification” refers to the removal of some or all of the lignan present in a lignin-containing sample. Delignification can be performed via chemical, mechanical, or enzymatic processes or combinations thereof.

“Oxygen delignification” refers to a delignification process wherein biomass (e.g., green liquor pretreated biomass) is contacted with oxygen gas in a pressurized vessel at an elevated temperature in an alkaline environment. Oxygen delignification is used in the paper industry to treat paper pulp in part to reduce the consumption of bleaching chemicals.

The term “refining” refers to a mechanical process of treating lignocellulosic-containing solids in order to beat, bruise, cut, and/or fibrillate the fibers therein. Thus, refining can be used to reduce lignocellulosic-containing solids in size as well as to providing material comprising bundles of cellulosic fibers, separate cellulosic fibers, fragments of cellulosic fibers, and combinations thereof.

The term “enzyme” refers to a protein that catalyzes the conversion of one molecule into another. The term “enzyme” as used herein includes any enzyme that can catalyze the transformation of a biomass-derived molecule to another biomass-derived molecule. In particular, enzymes include those which can degrade or otherwise transform saccharide, cellulose, or lignocellulose molecules to provide fermentable sugars and/or alcohols.

For use in a process of the presently disclosed subject matter, an enzyme can be specifically selected based on the particular end product desired from the biomass. The enzyme can also be selected to provide a desired property to a hydrolysis mixture. For example, an enzyme can be selected in order to produce a hydrolysis mixture of desired viscosity or pH.

The terms “lignocellulytic enzyme” “lignocellulose-processing enzyme”, and “lignocellulose-hydrolyzing enzyme” refer to enzymes that are involved in the disruption and/or degradation of lignocellulose. The disruption of lignocellulose by lignocellulytic enzymes leads to the formation of substances including monosaccharides, disaccharides, polysaccharides and phenols.

Lignocellulytic enzymes include, but are not limited to, cellulases, hemicellulases and ligninases. Thus, lignocellulytic enzymes include saccharification enzymes, i.e., enzymes which hydrolyze (i.e., depolymerize) polysaccharides. Saccharification enzymes and their use in biomass treatments have been previously reviewed. See Lynd, L. R., et al., Microbiol. Mol. Biol. Rev., 66, 506-577 (2002).

The term “cellulase” when used generally can refer to enzymes involved in cellulose degradation. Cellulase enzymes are classified on the basis of their mode of action. There are two basic kinds of cellulases: the endocellulases, which cleave polysaccharide polymer chains internally; and the exocellulases, which cleave from the reducing and non-reducing ends of molecules generated by the action of endocellulases. Cellulases include cellobiohydrolases, endoglucanases, and β-D-glucosidases. Endoglucanases randomly attack the amorphous regions of cellulose substrates, yielding mainly higher oligomers. Cellulobiohydrolases are exocellulases which hydrolyze crystalline cellulose and release cellobiose (glucose dimer). Both types of enzymes hydrolyze-1,4-glycosidic bonds. β-D-glucosidases or cellulobiase converts oligosaccharides and cellubiose to glucose.

As used herein, the term “cellulase” is typically used more specifically to refer to the enzyme is cellulase (E.C. 3.2.1.4), also known as an endoglucanase, which catalyzes the hydrolysis of 1,4-β-D-glycosidic linkages. The cellulase can be of microbial origin, such as derivable from a strain of a filamentous fungus (e.g., Aspergillus, Trichoderma, Humicola, Fusarium). Commercially available cellulase preparations which can be used include, but are not limited to, CELLUCLAST™, CELLUZYME™, CEREFLO™, and ULTRAFLO™ (available from Novozymes NS, Bagsvaerd, Denmark), SPEZYME™ CE and SPEZYME™ CP (available from Genencor International, Inc., Rochester, N.Y., United States of America) and ROHAMENT® CL (available from AB Enzymes GmbH, Darmstadt, Germany).

Hemicellulases are enzymes that are involved in hemicellulose degradation. Hemicellulases include xylanases, arabinofuranosidases, acetyl xylan esterases, glucuronidases, mannanases, galactanases, and arabinases. Similar to cellulase enzymes, hemicellulases are classified on the basis of their mode of action: the endo-acting hemicellulases attack internal bonds within the polysaccharide chain; the exo-acting hemicellulases act progressively from either the reducing or non-reducing end of polysaccharide chains. More particularly, endo-acting hemicellulases include, but are not limited to, endoarabinanase, endoarabinogalactanase, endoglucanase, endomannanase, endoxylanase, and feraxan endoxylanase. Examples of exo-acting hemicellulases include, but are not limited to, α-L-arabinosidase, β-L-arabinosidase, α-1,2-L-fucosidase, α-D-galactosidase, β-D-galactosidase, β-D-glucosidase, β-D-glucuronidase, β-D-mannosidase, β-D-xylosidase, exo-glucosidase, exo-cellobiohydrolase, exo-mannobiohydrolase, exo-mannanase, exo-xylanase, xylan α-glucuronidase, and coniferin β-glucosidase.

Ligninases are enzymes that are involved in the degradation of lignin. A variety of fungi and bacteria produce ligninases. Lignin-degrading enzymes include, but are not limited to, lignin peroxidases, manganese-dependent peroxidases, hybrid peroxidases (which exhibit combined properties of lignin peroxidases and manganese-dependent peroxidases), and laccases. Hydrogen peroxide, required as a co-substrate by the peroxidases, can be generated by glucose oxidase, aryl alcohol oxidase, and/or lignin peroxidase-activated glyoxal oxidase.

The terms “hydrolyze,” and variations thereof refer to the process of converting polysaccharides (e.g., cellulose) to fermentable sugars, e.g., through the hydrolysis of glycosidic bonds. This process can also be referred to as saccarification. Hydrolysis can be effected with enzymes or chemicals. Enzymes can be added to biomass directly (e.g., as a solid or liquid enzyme additive) or can be produced in situ by microbes (e.g., yeasts, fungi, bacteria, etc.). Hydrolysis products include, for example, fermentable sugars, such as glucose and other small (low molecular weight) oligosaccharides such as monosaccharides, disaccharides, and trisaccharides. Hydrolysis products can also simply include lower molecular weight polysaccharides than those in the original cellulose or lignocellulose. “Suitable conditions” for saccharification refer to various conditions including pH, temperature, biomass composition, and enzyme composition.

The term “filter paper unit” (or FPU) refers to the amount of enzyme required to liberate 2 mg of reducing sugar (e.g., glucose) from a 50 mg piece of Whatman No. 1 filter paper in 1 hour at 50° C. at approximately pH 4.8.

“Fermentation” or “fermenting” can refer to the process of transforming an organic molecule into another molecule using a micro-organism. For example, “fermentation” can refer to transforming sugars or other molecules from biomass to produce alcohols (e.g., ethanol, methanol, butanol); organic acids (e.g., citric acid, acetic acid, itaconic acid, lactic acid, gluconic acid); ketones (e.g., acetone), amino acids (e.g., glutamic acid); gases (e.g., H₂ and CO₂), antibiotics (e.g., penicillin and tetracycline); enzymes; vitamins (e.g., riboflavin, B₁₂, beta-carotene); and/or hormones. Thus, fermentation includes alcohol fermentation. Fermentation also includes anaerobic fermentations.

Fermenting can be accomplished by any organism suitable for use in a desired fermentation step, including, but not limited to, bacteria, fungi, archaea, and protists. Suitable fermenting organisms include those that can convert mono-, di-, and trisaccharides, especially glucose and maltose, or any other biomass-derived molecule, directly or indirectly to the desired fermentation product (e.g., ethanol, butanol, etc.). Suitable fermenting organisms also include those which can convert non-sugar molecules to desired fermentation products.

In some embodiments, the fermenting is effected by a fungal organism (e.g., yeast or filamentous fungi). The yeast can include strains from a Pichia or Saccharomyces species. In some embodiments, the yeast can be Saccharomyces cerevisiae. In some embodiments, the fermenting is effected by bacteria. For example, the bacteria can be Clostridium acetobutylicum (e.g., when butanol is the desired fermentation product) or Corynebacterium glutamicum (e.g., when monosodium glutamate (MSG) is the desired fermentation product). In some embodiments, the micro-organism (e.g. yeast or bacteria) can be a genetically modified micro-organism. In some instances, the organism can be yeast or other organism having or modified to be active in the presence of high concentrations of alcohol.

Thus “fermentation” and grammatical variations thereof refer to the conversion of a fermentable sugar to an alcohol (e.g., methanol, ethanol, propanol, butanol, etc.). The particular product of a given alcohol fermentation can be determined by the biocatalyst used in the fermentation and/or the substrate of fermentation (i.e., the type of fermentable sugar being converted).

In certain embodiments, fermenting can comprise contacting a mixture including biomass-derived sugars with an alcohol-producing biocatalyst, such as a yeast or another alcohol-producing microbe. In some embodiments, fermenting involves simultaneous saccharification (e.g., hydrolysis) and fermentation (SSF). The amount of fermentation biocatalyst employed can be selected to effectively produce a desired amount of ethanol in a suitable time and/or upon the sugar content of a given fermentation mixture. The use of alcohol-producing biocatalyst can increase the rate of saccharification by reducing the concentration of sugars, which can inhibit saccharification biocatalysts.

“Suitable conditions” for alcohol fermentation can refer to conditions that support the production of ethanol or another alcohol by a biocatalyst. Such conditions can include pH, nutrients, temperature, atmosphere, and other factors.

“Dehydrating” refers to removing the residual water left in ethanol following distillation. The residual water is generally about 5% by volume. Dehydration can be performed using molecular sieves.

### II. HIGH FIBER CONSISTENCY ENZYMATIC HYDROLYSIS OF LIGNOCELLULOSIC BIOMASS

The presently disclosed subject matter provides methods of hydrolyzing lignocellulosic materials using enzymes (i.e., lignocellulose-degrading enzymes) at high fiber consistency, as well as methods of preparing alcohols from lignocellulosic biomass that involve enzymatic hydrolysis performed at high fiber consistency. In some embodiments, the methods involve mixing lignocellulose-hydrolyzing enzymes with cellulose fibers at a low concentration, thickening the mixture to increase the fiber content, and then hydrolyzing the fibers for a period of time at the increased fiber consistency. At high fiber concentrations, the amount of water in the hydrolysis mixture can be reduced, thereby increasing the subsequent concentration of fermentable sugars that are available for fermentation. Thus, the size of the equipment needed during fermentation of the sugars and recovery of the alcohol produced during fermentation is reduced. Therefore, while the overall conversion of biomass to fermentable sugars using high consistency enzymatic hydrolysis can result in lower levels of enzymatic hydrolysis of the biomass, the use of high consistency enzymatic hydrolysis can cut down on capital costs for the overall biomass-to-alcohol process.

In some embodiments, additional enzymes are added after the thickening step, after the actions of the first portion of enzymes have reduced cellulose fibers in size somewhat to decrease the viscosity of the hydrolysis mixture and make the homogeneous mixing of the additional enzymes easier. In some embodiments, the first portion of the enzymes includes cellulase, which can absorb well to the biomass and disrupt the crystalline structure of the cellulose in the fibers, exposing individual fibers for additional enzymatic action. In some embodiments, the presently disclosed subject matter further relates to methods of recycling lignocellulose-hydrolyzing enzymes. As enzyme costs, particularly for cellulase, can be quite high, re-cycling the enzymes can provide significant savings.

Thus, in some embodiments, the presently disclosed subject matter provides a method of producing an alcohol from a lignocellulosic biomass, wherein the method can comprise: providing lignocellulosic biomass; contacting the lignocellulosic biomass with a first enzyme composition for a first period of time to provide a first hydrolysis mixture; thickening the first hydrolysis mixture to from a second hydrolysis mixture; hydrolyzing the second hydrolysis mixture for a second period of time to provide a fermentable sugar mixture; and fermenting the fermentable sugar mixture to provide an alcohol.

In some embodiments, the lignocellulosic biomass (such as the as-harvested biomass) is pretreated to increase enzymatic digestability prior to enzymatic hydrolysis by lignocellulose-degrading enzymes and/or to make handling of the biomass easier. Pretreatments can be mechanical, chemical, or biochemical processes or combinations thereof. The pretreating can involve removing or altering lignin, removing hemicellulose, decrystallizing cellulose, removing acetyl groups from hemicellulose, reducing the degree of polymerization of cellulose, increasing the pore volume of lignocellulose biomass, increasing the surface area of lignocellulose, or any combination thereof. The pretreatment can comprise one or more technique known in the art of biomass-to-alcohol conversion, including, but not limited to, autohydrolysis, steam explosion, grinding, chopping, ball milling, compression milling, radiation, flow-through liquid hot water treatment, dilute acid treatment, concentrated acid treatment, peracetic acid treatment, supercritial carbon dioxide treatment, alkali treatment, organic solvent treatment, cellulose solvent treatment, and treatment with an aerobic fungi. The alkali treatment can include sodium hydroxide treatment, lime treatment, wet oxidation, ammonia treatment, and oxidative alkali treatment.

In some embodiments, the alkali treatment comprises green liquor (GL) treatment, as described in the co-pending PCT International Patent Application titled “Production of Ethanol from Lignocellulosic Biomass Using Green Liquor Pretreatment” (based on U.S. Provisional Patent Application Ser. No. 61/116,934). Green liquor treatment can involve treatment of biomass with an alkaline composition comprising sodium sulfide and sodium carbonate at a temperature of between about 100° C. to about 220° C. (e.g., about 100, 110, 120, 130, 140, 150, 160, 170, 180, 190, 200, 210, or 220° C.) for between about 0.25 and about 4 hours (e.g., about 0.25, 0.5, 0.75, 1.0, 1.5, 2.0, 2.5, 3.0, 3.5, or 4.0 hours) in a carbon steel pressure vessel. The charge of total titratable alkali provided by the green liquor can be between about 4% and about 25% or between about 12% and about 20% (e.g., 12%, 13%, 14%, 15%, 16%, 17%, 18%, 19%, or 20%). The sulfidity of the green liquor can be between about 5% and about 50% (e.g., about 5, 10, 15, 20, 25, 30, 35, 40, 45, or 50%). In some cases the sulfidity is about 25%.

In some embodiments, the pretreatment can comprise a washing step, to remove any solubilized lignin, unfermentable solubilized cellulose products or any chemicals used in a pretreatment step. In some embodiments, green liquor pretreated biomass can be further pretreated via oxygen delignification (e.g., treatment with oxygen gas in a pressurized vessel at a temperature of between about 60° C. and about 150° C. for between about 10 minutes to about 4 hours), refining to reduce the size of the solid materials and/or separate pulp fibers (e.g., with refining equipment, such as a disk refiner, a PFI mill, or any other refiner, such as those typically used to refine paper pulp in the paper industry) or a combination thereof. In some embodiments, the pretreatment (e.g., the green liquor pretreatment) further comprises the use of one or more additives to increase the yield of carbohydrate (e.g., cellulose and hemicellulose) during the pretreatment. Such additives can include, but are not limited to, anthraquinone and sodium polysulfides.

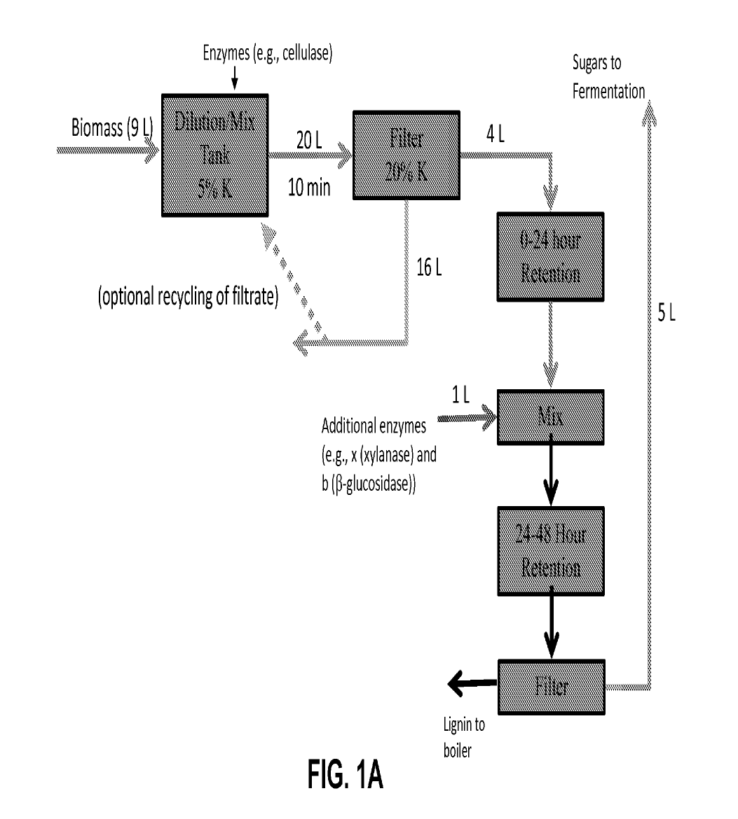

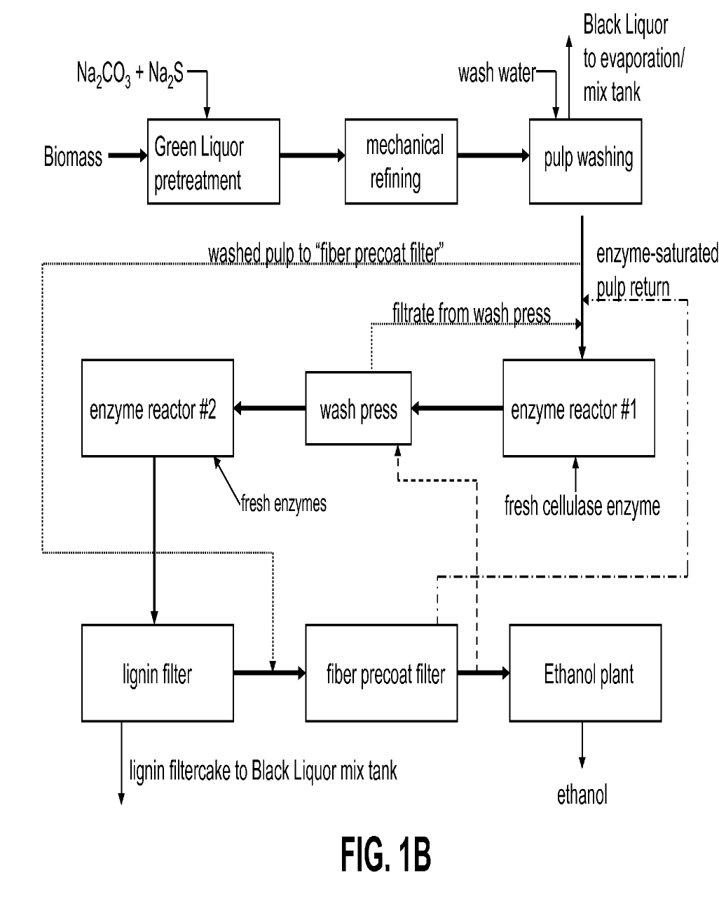

As shown in FIG. 1A, biomass (or pretreated biomass), such as hardwood or softwood chips, can be added to a dilution/mix tank and diluted (e.g., with water) to 5-10% fiber consistency (e.g., about 5, 6, 7, 8, 9 or 10% fiber consistency). In some embodiments, the fiber concentration is diluted to about 5%. A first enzyme composition comprising lignocellulose-hydrolyzing enzymes can be added and the mixture stirred for a period of time (e.g., between about 1 and 20 minutes). In some embodiments, the mixture can be stirred for about 5, 10, or 15 minutes. The amount of enzyme added can vary depending upon the type of biomass and/or pretreatment used. In some embodiments, between about 5 filter paper units (FPU) and about 85 FPU per gram of biomass material can be added (e.g., about 5, 10, 15, 20, 25, 30, 35, 40, 45, 50, 55, 60, 65, 70, 75, 80 or 85 FPU). The first enzyme composition can comprise cellulase, either alone, or in combination with other lignocellulase-hydrolyzing enzymes (e.g, xylanase and β-glucosidase). The cellulase can react quickly with the cellulose fibers, for example, to open physical pores for subsequent enzyme action.

The mixing and stirring can be done at any suitable temperature or pH to facilitate adsorption of the enzymes or enzymatic hydrolysis by the enzymes. In some embodiments, the temperature is between about 4° C. and about 70° C. In some embodiments, the temperature is between about 4° C. and about 50° C. (e.g., about 4, 10, 15, 20, 25, 30, 35, 38, 40, 42, 45, or 50° C.). In some embodiments, the temperature is about 38° C. In some embodiments, the temperature is about 50° C.

The pH can be optimized based on the type of enzymes being used. In some embodiments, the pH can be between about 4 and about 5 (e.g., about 4.0, 4.1, 4.2, 4.3, 4.4, 4.5, 4.6, 4.7, 4.8, 4.9, and 5.0). In some embodiments, the pH is about 4.8. The pH can be adjusted using pH-adjusting chemicals (e.g., acids, bases, buffers), so long as the pH-adjusting chemicals do not adversely affect the functioning of the enzymes.

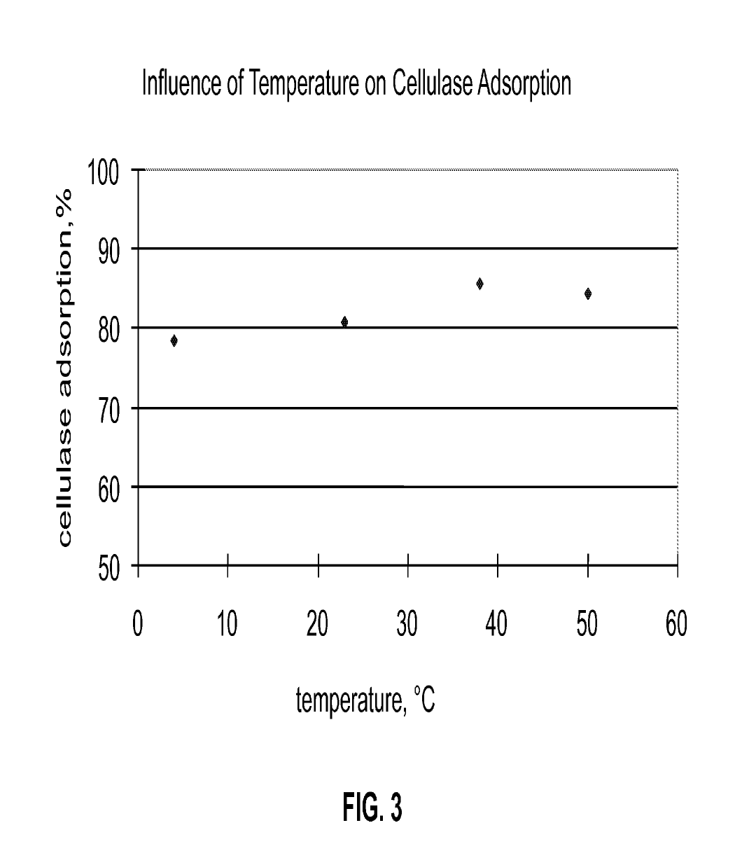

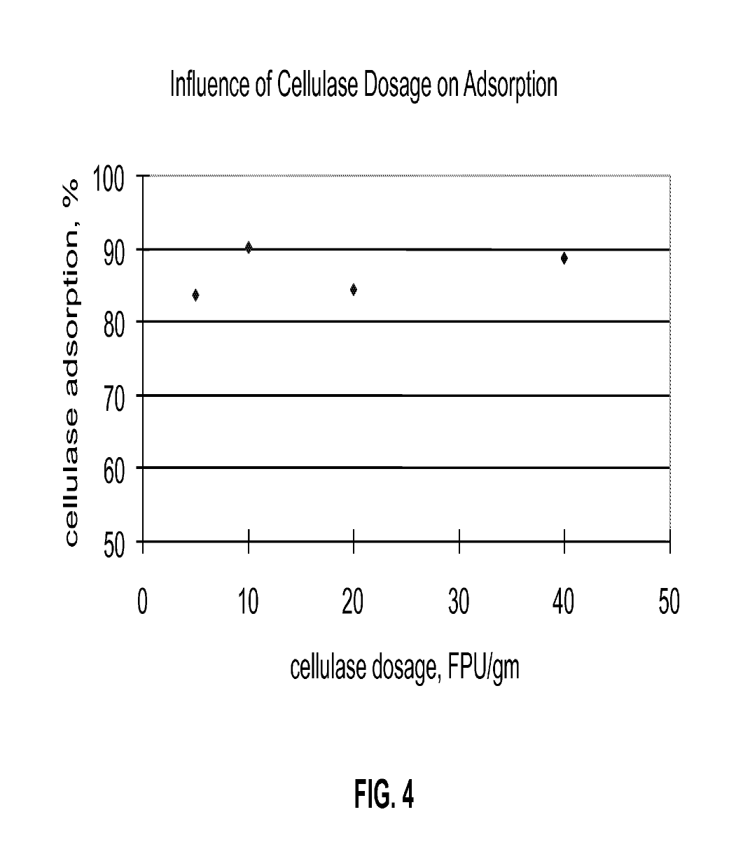

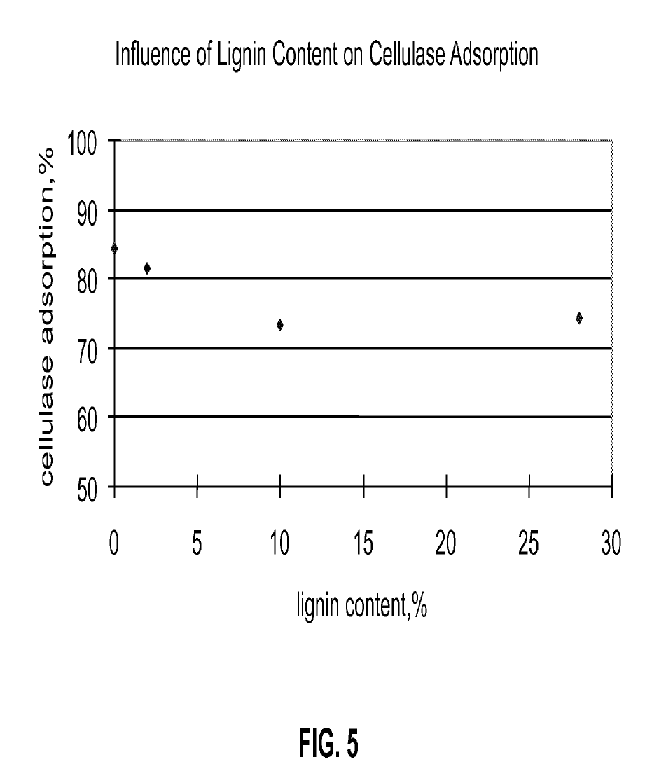

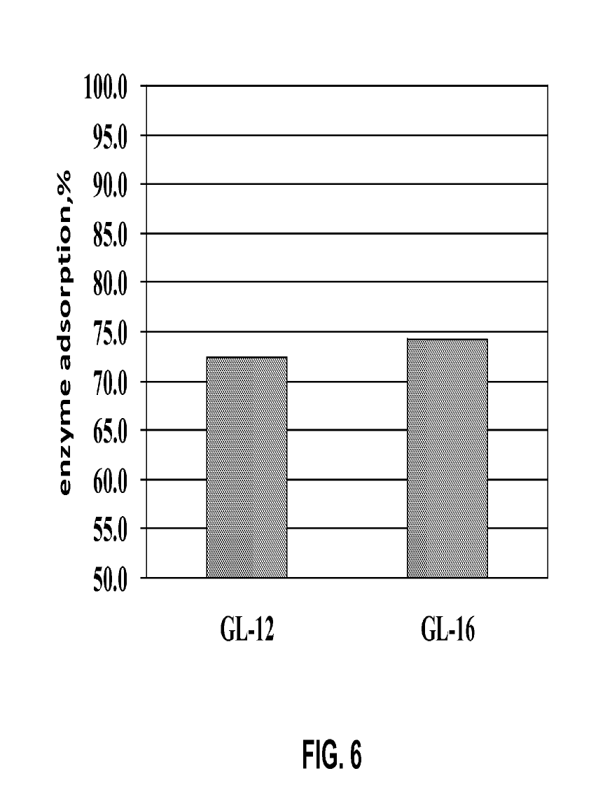

Referring now to FIGS. 3-6, effects of various pretreatments are evaluated in representative embodiments of the presently disclosed subject matter. These embodiments are also described in the Examples presented hereinbelow. FIG. 3 shows the effect of temperature on cellulase adsorption to the biomass. While the effects of temperature are not large, in the embodiments implemented for FIG. 3, the adsorption was observed to be best at about 38° C. FIGS. 4 and 5 show the effect of cellulase dosage and lignin content on enzyme adsorption. While enzyme dosage has minimal effects on adsorption, higher lignin content can reduce enzyme adsorption. FIG. 6 shows the effect of two different green liquor pretreatments on enzyme adsorption. GL-12 refers to green liquor pretreatment using an alkaline solution having a TTA of 12%. GL-16 refers to green liquor pretreatment using an alkaline solution having a TTA of 16%. As seen in FIG. 6, the effect of TTA on enzyme adsorption is minimal.

In some embodiments, after allowing the mixing and adsorption of the enzymes from the first enzyme composition, the mixture can be thickened to between about 15% and about 30% K (e.g., about 15%, 16%, 17%, 18%, 19%, 20%, 21%, 22%, 23%, 24%, 25%, 26%, 27%, 28%, 29%, or 30% K). In some embodiments, the mixture is thickened to about 20% K. The thickening can be done by any suitable technique. In some embodiments, the thickening can be done by either gravity or vacuum filtration, for example, using a filter press.

When the first hydrolysis mixture is thickened from about 5% K to about 20% K, the filtrate can comprise about 80% of the total volume from the first hydrolysis mixture. See FIG. 1A, which relates to an example where 4 liters (L) of an original 20 L 5% K hydrolysis mixture is left after filtration and carried on into further hydrolysis, while the filtrate comprises the remaining 16 L, some of which can optionally be feed back into the dilution/mix tank to dilute new incoming batches of biomass, which can comprise about 9 L of material straight from any pretreatment processing. In addition to water, the filtrate can also comprise some cellulose fibers and non-adsorbed enzymes, which, if desired can be fed back into the system in the filtrate to dilute biomass, thereby reusing the unabsorbed enzyme. While cellulase adsorption can be reasonably high (e.g., between about 80 and 90%), other enzymes, such as xylanase, do not tend to adsorb well to fibers and generally are removed during thickening and can be present in the filtrate. Thus, it can be cost effective to add cellulase prior to the thickening step, and to add other enzymes later, as part of a second enzyme composition, though this is not necessarily required. Reference to particular volumes in FIG. 1A is for purposes of illustration only, and not limitation.

Generally, the filtrate reuse described herein relates to methods wherein the main enzymatic hydrolysis of the fibers is performed at fiber consistencies above 10% K. However, in some embodiments, it can be advantageous to filter the enzyme/fiber mixture and reuse the filtrate and, in some cases, some of the fibers, to dilute fresh biomass or pretreated biomass, thereby reusing the enzymes, even if the main hydrolysis is to be performed at more typical fiber consistencies of between 5-10%. Reusing the filtrate can increase overall conversion of cellulose to sugar from 63-65% to up to 70% and above.

Enzymatic hydrolysis of the thickened mixture can proceed for any suitable time and temperature to provide sufficient fermentable sugars. In some embodiments, hydrolysis can proceed for between about 2 hours and about 3 days (e.g., about 2, 4, 6, 8, 10, 12, 14, 16, 18, 20, 24, 28, 32, 36, 40, 44, 48, 52, 56, 60, 64, 68, or 72 hours). In some embodiments, enzymatic hydrolysis in the thickened mixture is carried out at about 50° C.

In some embodiments, a second enzyme composition (e.g., the “additional enzymes” in FIG. 1A) is added at some time during the hydrolysis. The addition of a second enzyme composition can be referred to as split enzyme dosing. In some embodiments, such as that shown in FIG. 1A, the second enzyme composition is added between about 0 hours (i.e., immediately after thickening) and about 24 hours following thickening (i.e., at 0, 1, 2, 3, 4, 5, 6, 7, 8, 9, 10, 11, 12, 13, 14, 15, 16, 17, 18, 19, 20, 21, 22, 23, or 24 hours following thickening). Thus, in some embodiments, as shown in FIG. 1A, there can be a 0-24 hour retention of the originally thickened mixture prior to the addition of further enzymes, during which enzymatic hydrolysis can take place due to the presence of enzymes added prior to the thickening. In some embodiments, the second enzyme composition is added (e.g., in a “Mix” step as shown in FIG. 1A) between about 2 hours and about 3 hours following thickening.

The second enzyme composition can comprise xylanase (i.e., x in FIG. 1A), β-glucosidase (i.e., b in FIG. 1A), combinations of x and b, and/or other lignocellulosic-hydrolyzing enzymes. All the enzyme or enzymes of the second enzyme composition can be added together, or portions of the second enzyme composition (e.g., an aliquot of the total second enzyme composition or each different type of enzyme) can be added sequentially over a period of time (e.g., a few minutes or hours apart).

In some embodiments, the second enzyme composition comprises at least some additional cellulase (i.e., cellulase in addition to that remaining from the first enzyme composition). For example, the first enzyme composition can comprise between about 25% and about 50% of the total cellulase dose, while the second enzyme composition can comprise the remainder of the cellulase dose (e.g, about 50%, about 55%, about 60%, about 65%, about 70% or about 75%). In some embodiments, split enzyme dosing can refer to adding a portion of cellulase prior to thickening and a portion of cellulase after thickening.

If necessary, additional diluent can be added to assist in the mixing process at this stage. Thus, in some embodiments, the second enzyme composition can comprise some additional water or filtrate from the thickening step. In some embodiments, the diluent can include a sugar solution prepared from the enzymatic hydrolysis of another batch of biomass. In the non-limiting example shown in FIG. 1A, the second enzyme composition of additional enzymes has a volume of about 1 L.

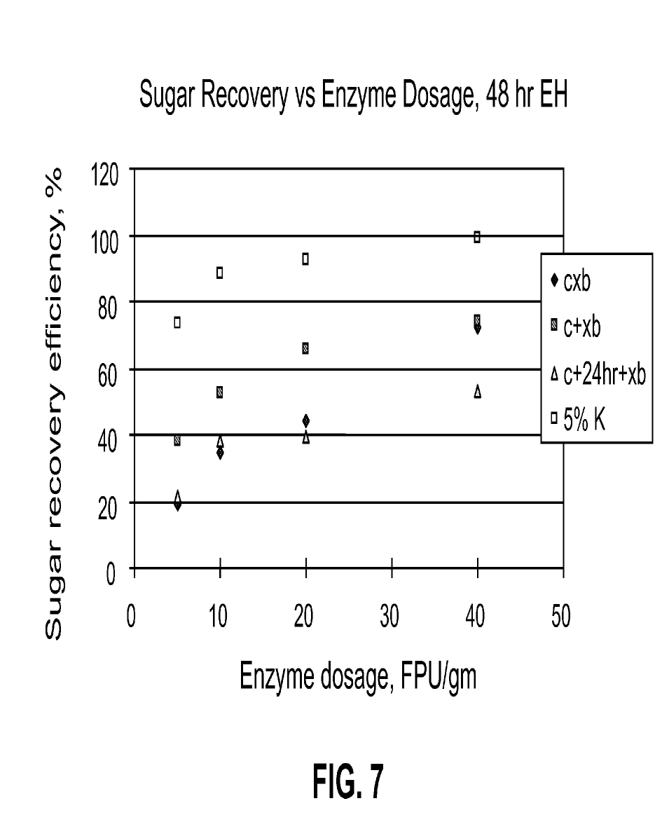

FIG. 7 shows the effect of total enzyme dosage and both single enzyme composition dosing and split enzyme dosing on sugar recovery efficiency in exemplary, non-limiting embodiments of the presently disclosed subject matter, also described in the Examples hereinbelow. Following the addition of the second enzyme composition, the enzymatic hydrolysis of the mixture is allowed to continue for a period of time. The period of time can range between about 2 hours and about 72 hours. In some embodiments, the period of time can range between about 24 and about 48 hours (e.g., about 24, 26, 28, 30, 32, 34, 36, 38, 40, 42, 44, 46, or 48 hours). Thus, as shown in FIG. 1A, there can be a 24-48 hour retention step following the mix step.

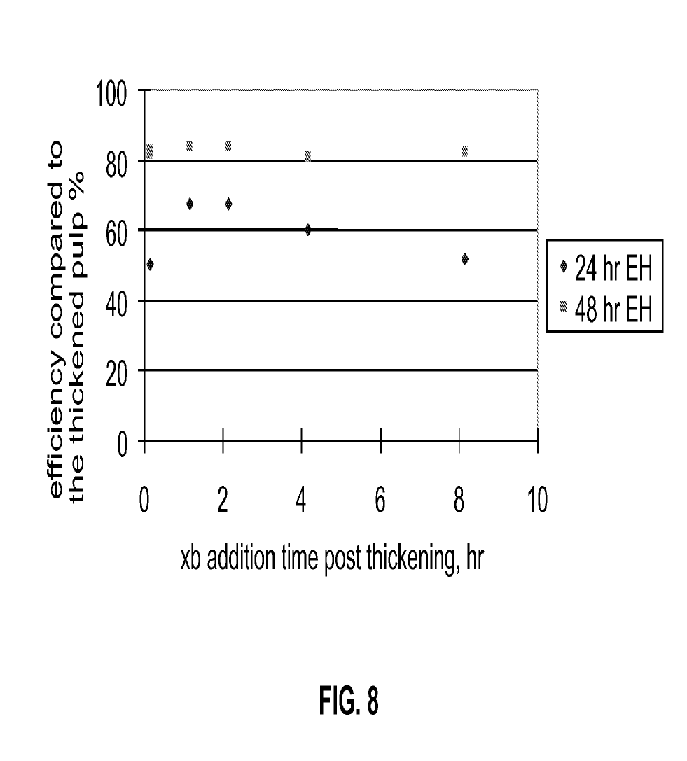

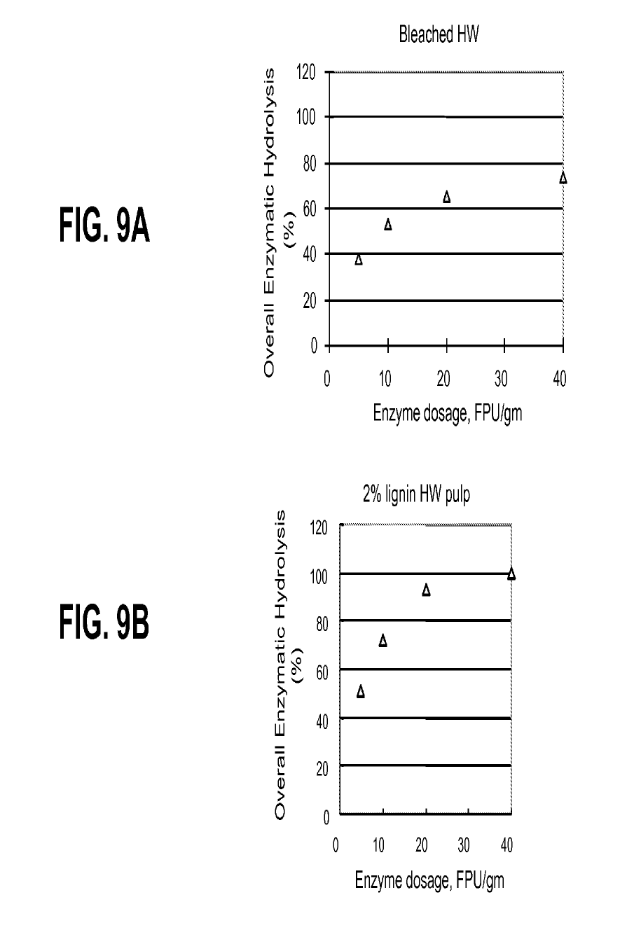

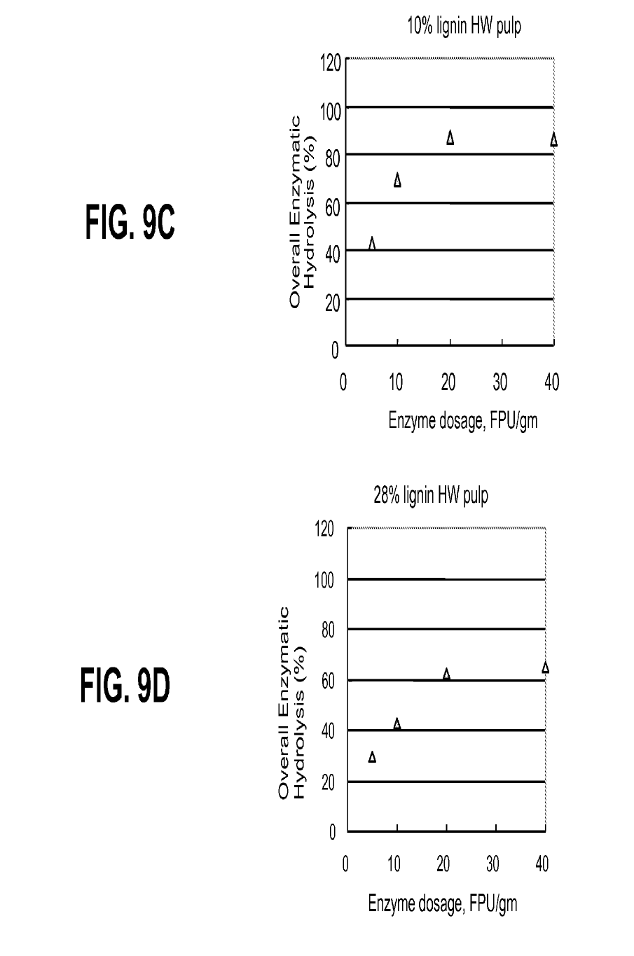

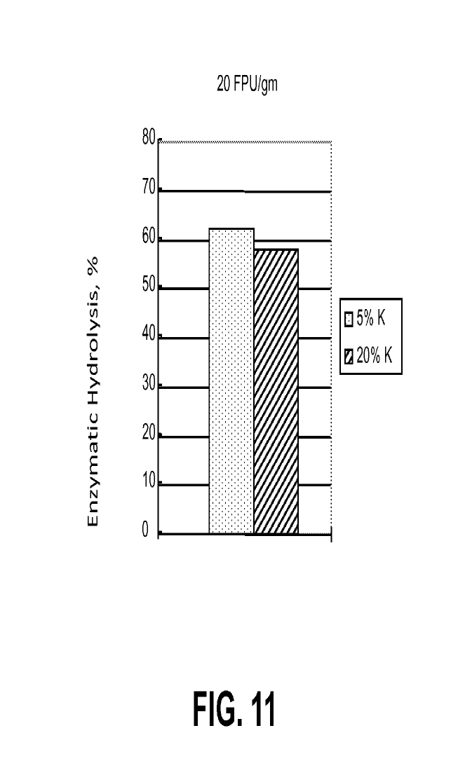

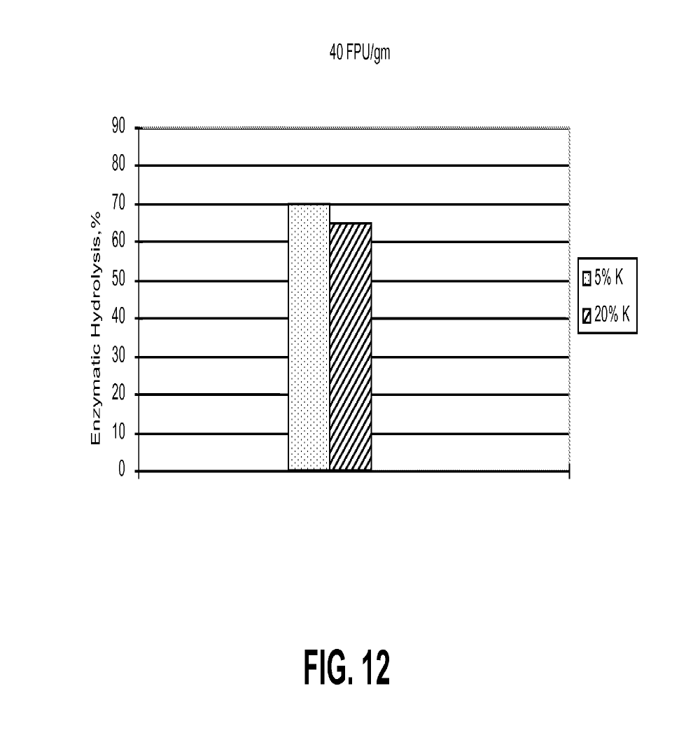

FIG. 8 shows the effects of adding a second enzyme composition comprising xylanase and β-glucosidase at between 2 and 8 hours following thickening and allowing the hydrolysis to continue for a further 24 or 48 hours in exemplary, non-limiting embodiments of the presently disclosed subject matter, also described in the Examples hereinbelow. FIGS. 9A-9D and 10-15 provide additional data regarding enzymatic hydrolysis efficiency in mixtures thickened to 20% K, in mixtures thickened to 20% K as compared to mixtures hydrolyzed at 5% K, and of various split enzyme dosing schedules according to exemplary, non-limiting embodiments of the presently disclosed subject matter, also described in the Examples hereinbelow. In some cases, enzymatic efficiency at high consistency is lower than at the typical 5% K used; however, capital saving costs can outweigh this yield loss. With split enzyme dosages, enzymatic efficiency at higher consistency can be about the same as that for 5% K hydrolysis reactions. In some embodiments, the hydrolysis efficiency of the method can be about 60%, 65%, 70%, 75%, 80% or higher. In some embodiments, the hydrolysis efficiency can be about 84%.

The fermentable sugar mixture resulting from hydrolysis can comprise about 6% or more fermentable sugar by volume, instead of the 3-4% usually found following hydrolysis of 5% K mixtures. In some embodiments, the fermentable sugar mixture can comprise between about 10% and about 15% fermentable sugar by volume (e.g., about 10%, 11%, 12%, 13%, 14%, or 15%). In some embodiments, the fermentable sugar mixture comprises about 12% fermentable sugar by volume.

After hydrolysis is complete, the mixture is filtered to remove lignin, which can be burned as a fuel, and a sugar solution, which can be fermented with a suitable microorganism (e.g., yeast or another alcohol-producing microbe) as described herein to provide an alcohol. In the non-limiting example shown in FIG. 1A, filtration of the hydrolysis mixture results in the collection of a sugar solution having a volume of about 5 L.

In some cases part of the sugar solution can be re-used to dilute the first hydrolysis mixture or to dilute the second hydrolysis mixture following addition of the second part of a split enzyme dose. The sugar solution can also be used as a source of sugar, for example, in the food industry. Thus, in some embodiments, the presently disclosed subject matter can provide a method of producing sugar from a lignocellulosic biomass, wherein the method comprises: providing lignocellulosic biomass, contacting the lignocellulosic biomass with a first enzyme composition for a first period of time to provide a first hydrolysis mixture, thickening the first hydrolysis mixture to form a second hydrolysis mixture; and hydrolyzing the second hydrolysis mixture for a second period of time to provide a sugar mixture.

In embodiments wherein the sugar mixture is fermented, the alcohol provided by the fermenting can be ethanol. Based on a 10-15% sugar content, the alcohol mixture formed during fermentation can include between about 5% and about 7.5% alcohol (e.g., about 5%, about 5.5%, about 6.0%, about 6.5%, about 7.0%, or about 7.5% alcohol). Distillation and additional dehydration of the alcohol mixture (e.g., using molecular sieves or another suitable hygroscopic material) can provide 95% or greater alcohol (e.g., ethanol) solutions. If the alcohol is ethanol, the ethanol can be denatured, if desired, through the addition of a suitable additive (e.g., methanol, isopropyl alcohol, acetone, methyl ethyl ketone, or methyl isobutyl ketone). The alcohol can be used directly as a fuel or for another purpose, or can be mixed with another component to provide a fuel mixture. For example, the ethanol produced by one of the presently disclosed methods can be mixed with gasoline to provide a gasohol. Thus, in some embodiments, the presently disclosed subject matter provides a method of producing a biofuel.

FIG. 1B shows a scheme for the production of ethanol from biomass according to an exemplary embodiment of the presently disclosed subject matter. As illustrated in FIG. 1B, biomass (e.g., wood chips) can be pretreated with green liquor (or another pretreatment). Following pretreatment, the pretreated biomass can undergo an optional mechanical refining step (e.g., using a disc refiner or other mechanical refiner known in the art of paper manufacturing). The mechanical refining can reduce the size of the pretreated chips and/or the size of the wood fibers or fiber bundles. Refining can also separate fiber bundles. In some embodiments, the pretreated biomass can comprise single fibers or mainly single fibers.

Then, as shown in FIG. 1B, the pretreated biomass can be washed (e.g., with water). The washing can remove the “black liquor” resulting from the green liquor pretreatment. The black liquor can comprise alkaline chemicals, solubilized lignin, and solubilized (but unfermentable) cellulose-derived molecules. The washed biomass can then be fed into a first enzyme reactor (i.e., enzyme reactor #1, which can correspond to the dilution/mix tank of FIG. 1A).

Continuing with FIG. 1B, lignocellulase-hydrolyzing enzyme (e.g., fresh cellulase) can be added to the first enzyme reactor. Typically, pulp coming from the pulp washing step can have a fiber consistency of about 14%. Thus, it will generally (but not necessarily) need to be diluted to provide efficient mixing with the enzyme. Optionally, liquid to dilute the biomass in the first enzyme reactor can include filtrate from the wash press (i.e., filtrate resulting from thickening the biomass mixture from the first enzyme reactor following adsorption of the enzymes). Dilution liquid can also include other liquid, such as fresh water (i.e., water newly introduced into the biomass-to-ethanol process). Biomass can reside in the first enzyme reactor from between about 1 and about 20 minutes, for example, prior to thickening in the wash press.

Following thickening in the wash press (to between about 10% and about 30% K), the thickened biomass mixture can be introduced to a second enzyme reactor (i.e., enzyme reactor #2 of FIG. 1B) and allowed to hydrolyze for a period of time (typically about 1 to 3 days). The second enzyme reactor can be the same physical vessel as the first enzyme reactor, or can be a different vessel. Optionally, additional enzymes can be added as described hereinabove. Mixing of additional enzymes can be assisted as necessary by diluting the thickened mixture with, for example, reserved filtrate from the wash press, portions of filtered sugar solutions from prior hydrolysis mixtures, or fresh water. Generally any diluent used at this step will be no more than about 10% or 5% or less of the volume of the thickened mixture coming from the wash press. Following mixing of additional enzymes the contents of the second enzyme reactor can be allowed to hydrolyze without further mixing (e.g, stirring or other agitation), if desired.

Continuing with reference to FIG. 1B, after enzymatic hydrolysis in the second enzyme reactor, the hydrolyzed mixture can be filtered. For example, the hydrolyzed mixture can be introduced into a lignin filter to remove remaining solids, which can contain lignin. Optionally, the lignin filtercake can be added into a mix tank with the black liquor from the pulp washing step and burned to provide energy. The energy can be used, for example, to heat an enzyme reactor, during the distillation process, and/or during another step of the biomass-to-ethanol process. The energy from the lignin burning can also be used to fuel any external (i.e., non-biomass-to-ethanol) process.

In some embodiments, the sugar-containing filtrate from the lignin filter can optionally be filtered through a fiber precoat filter, as shown in FIG. 1B. The fiber precoat filter can be coated with some of the biomass (e.g., up to about 20%) from the pulp washing step. The pulp coated in the filter can adsorb remaining lignocellulase-hydrolyzing enzymes present in the sugar-containing filtrate. The pulp coating the fiber precoat filter can then be reintroduced into the process by being added as part of the pulp fed into the first enzyme reactor during a subsequent biomass conversion to reuse the readsorbed enzymes. The sugar-containing filtrate can be fermented, for example in a conventional ethanol plant, to provide ethanol.

### EXAMPLES

The following Examples have been included to provide illustrations of the presently disclosed subject matter. In light of the present disclosure and the general level of skill in the art, those of skill will appreciate that the following Examples are intended to be exemplary only and that numerous changes, modifications and alterations can be employed without departing from the spirit and scope of the presently disclosed subject matter.

### General Methods

Enzymatic hydrolysis sugar yields and wood and/or pulp polysaccharide and lignin contents can be measured by any suitable method, as would be readily understood by one of ordinary skill in the art upon review of the instant disclosure. Such measurements can be performed, for instance, according to analytical procedures available from the National Renewable Energy Laboratory (NREL, Golden, Colo., United States of America) and/or the Technical Association of the Pulp and Paper Industry (TAPPI, Norcross, Ga., United States of America), among others. For example, polysaccharide content in a wood or pulp sample can be measured by sulfuric acid hydrolysis of given amount of a pulp or wood sample, followed by analysis of the resulting sugars, to calculate the amount of corresponding polysaccharide originally present in the wood or pulp. In some embodiments, enzymatic hydrolysis efficiencies can be calculated based on solids weight loss. In some embodiments, enzymatic hydrolysis efficiencies can be calculated by comparing sugar yield to pulp or wood polysaccharide content.

### Example 1

### Enzyme Hydrolysis at High Consistency

Hardwood chips were pretreated with green liquor (12% or 16% TTA) at 160° C. as briefly described hereinabove, refined in a disc refiner and washed. The pretreated chips were then diluted with water to a fiber consistency of 5%, 7.5% or 10%. 20 FPUs/gram wood was added and the mixtures allowed to hydrolyze for up to 48 hours. At 6, 12, 24, or 48 hours, the mixtures were analyzed for monomeric sugar content (glucose, xylose, and mannose). The amount of sugars produced was compared to the amount of sugar theoretically present based on analysis of the original wood polysaccharide content to determine the percentage of total sugar yield.

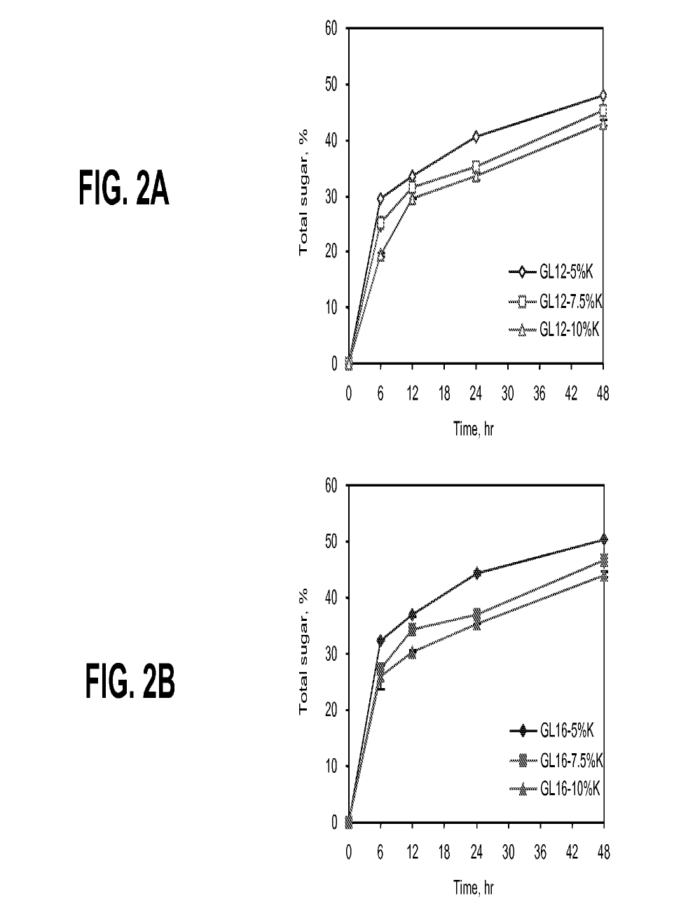

Results are shown in FIG. 2A for the chips pretreated with green liquor at 12% TTA (GL-12) and in FIG. 2B for the chips pretreated with green liquor at 16% TTA (GL-16). As shown in FIGS. 2A and 2B, increasing the fiber consistency can reduce the amount of sugar produced during enzymatic hydrolysis. Without being bound to any one theory, it is believed that this decrease can be due, at least in part, to inefficient mixing of the pulp and enzyme at the higher consistencies.

### Example 2

### Enzyme Adsorption

To offset some of the reduction in sugar production at higher consistencies, enzymes can be premixed with biomass at a lower consistency and the premixed material can be filtered to produce a thickened biomass mixture. However, since some benefits to premixing can be lost if substantial (i.e., >50%) of the premixed enzyme are lost during the thickening process (e.g., in the filtrate), the adsorption characteristics of enzymes to biomass fibers can help to determine which enzymes to use during a premix step.

A variety of cellulase/biomass mixtures were prepared to study the affects of temperature, enzyme dosage, and lignin content on cellulase adsorption. Bleached softwood or hardwood pulp was mixed with water and cellulase to provide mixtures having a fiber consistency of 5%. The mixtures were incubated for 10 minutes and vacuum filtered (using a filter press) to increase the fiber consistency to 20%. The filtrate was then analyzed for free (i.e., non-adsorped) cellulase. Cellulase adsorption was calculated as 100%-(amount of enzyme in the filtrate/amount of enzyme added to the pulp). Mixtures were also prepared using hardwood pulps with 2%, 10%, or 28% lignin and using green liquor pretreated pulps. Enzyme dosages used included 5, 10, 20, and 40 FPU. Incubation temperatures included 4, 23, 28, and 50° C.

Generally, between 75 and 90% of the cellulase was adsorbed to the wood pulp. Varying enzyme dosage at this level appeared to have little effect on adsorption. See FIG. 4. Of the different incubation temperatures studied, 38° C. provided the highest adsorption. See FIG. 3. Lignin content appeared to have some affect on enzyme adsorption in this Example. See FIG. 5. Generally, the higher the lignin content, the lower the enzyme adsorption. Varying green liquor pretreatment conditions from 12% (GL-12) to 16% (GL-16) TTA did not appear to greatly affect enzyme adsorption. See FIG. 6.

Xylanase adsorption to bleached hardwood pulp (0% lignin) was studied in a similar manner, using xylanase dosages of between 1 and 10 FPU. In contrast to cellulase, xylanase does not appear to become adsorbed to the pulp. The amount of xylanase that was found in the filtrate was proportional to the amount of hydrolysis mixture liquid that was in the filtrate.

### Example 3

### Effects of Increased Consistency Following Enzyme Addition

Bleached hardwood pulp was added to a dilution/mixing tank and diluted to 5% K with water. Cellulase, xylanase, and β-glucosidase were added at a desired dosage and the mixture stirred for 10 minutes. Then the mixture was thickened by vacuum filtration such that the thickened mixture had a consistency of 20%. Enzyme hydrolysis was allowed to continue for 48 hours after which time the sugar content of the hydrolysis mixture was analyzed. As a control, sugar contents of un-thickened mixtures dosed with the same amounts of cellulase were also analyzed after 48 hours.

In addition, the affect of adding further enzymes after thickening was studied. Pulp mixtures that had been incubated for 10 minutes at 5% K with cellulase alone were thickened to 20% K using vacuum filtration. Xylanase and β-glucosidase was added to the thickened mixture immediately and the mixture allowed to hydrolyze for 48 hours prior to sugar analysis. Alternatively, the thickened mixture was hydrolyzed for 24 hours and then the additional enzymes were added and then the mixture was hydrolyzed for another 24 hours.

FIG. 7 shows the sugar recovery efficiency results from the various samples. Enzymatic hydrolysis at 20% K was somewhat less efficient than at 5% K. At an enzyme dosage of 20 FPU/gram, the sugar recovery from higher consistency mixtures was about 70-80% of the sugar recovery from the mixture hydrolyzed at 5% K. Adding the xylanase and β-glucosidase immediately following thickening led to greater enzymatic efficiency than when all the enzymes were added together prior to thickening or than when the xylanase and β-glucosidase were added 24 hours after thickening.

### Example 4

### Additional Enzyme Addition Timing

The effect of xylanase/β-glucosidase addition time was studied further. As in Example 3, bleached hardwood pulp was placed in a dilution/mixing tank and diluted to 5% K with water. Cellulase (20 FPU/gram pulp) was added and the resulting mixture incubated for 10 minutes. The mixture was thickened using vacuum filtration to increase the fiber consistency to 20% K. Hydrolysis was then allowed to proceed for 15 minutes to 10 hours prior to addition of xylanase and β-glucosidase. Following the adding of the hemicellulose-degrading enzymes, hydrolysis was continued for a further 24 or 48 hours.

FIG. 8 shows the effect of varying the addition time of the hemicellulose-degrading enzymes. Waiting to add the additional enzymes for 2-3 hours appeared to provide the highest enzymatic efficiency, particularly over shorter total hydrolysis times.

### Example 5

### Effect of Lignin Content on Enzymatic Hydrolysis at High Consistency

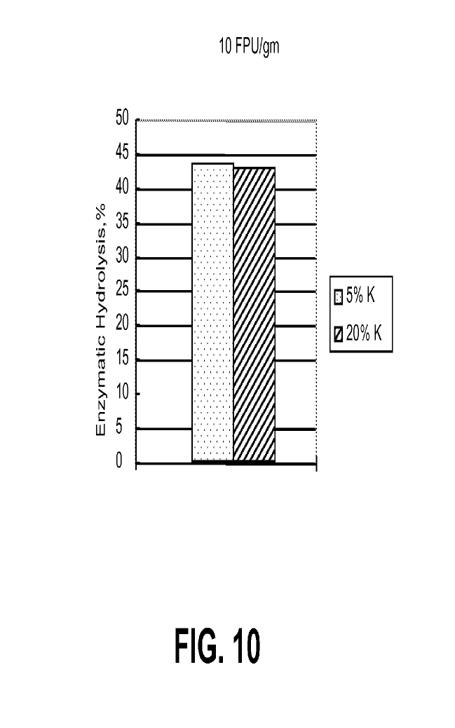

In order to evaluate the effect of lignin of enzymatic hydrolysis efficiency at high consistency, four different types of hardwood pulp were subjected to enzymatic hydrolysis: bleached hardwood pulp (0% lignin) and hardwood pulps having 2%, 10%, or 28% lignin. Enzymatic hydrolysis of the pulps was carried out at 5% K for 48 hours at various enzyme dosages (5, 10, 20, or 40 FPU/gram) using an enzyme mixture containing cellulase, xylanase and β-glucosidase as a control. Enzymatic hydrolysis of the four different pulps was also performed at 20% K. Briefly, cellulase only was added to the pulp at 5% K and incubated for 10 minutes prior to thickening to 20% K. The thickened mixture was allowed to hydrolyze for 2 hours prior to addition of xylanase and β-glucosidase. After addition of the hemicellulose-degrading enzymes the mixtures were allowed to hydrolyze for an additional 46 hours. The hydrolysis mixtures were filtered and the amounts of monomeric sugars in the filtrates were determined in order to calculate the percentage of enzymatic hydrolysis that had occurred. Results for the samples wherein enzymatic hydrolysis was carried out at 20% K are provided in FIGS. 9A-9D. The enzymatic hydrolysis percentages from samples having 28% lignin content that were hydrolyzed with 10, 20, and 40 FPU/gram enzyme dosages at 20% K are compared to the corresponding control samples hydrolyzed at 5% K in FIGS. 10-12.

### Example 6

### Split Addition of Cellulase

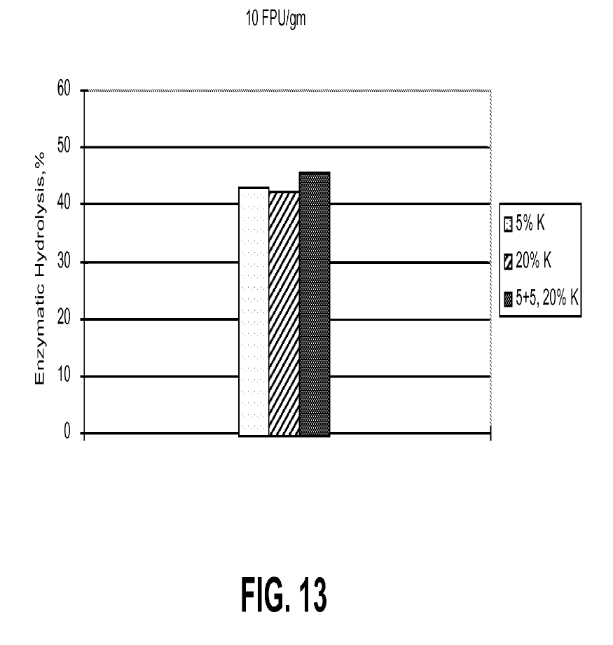

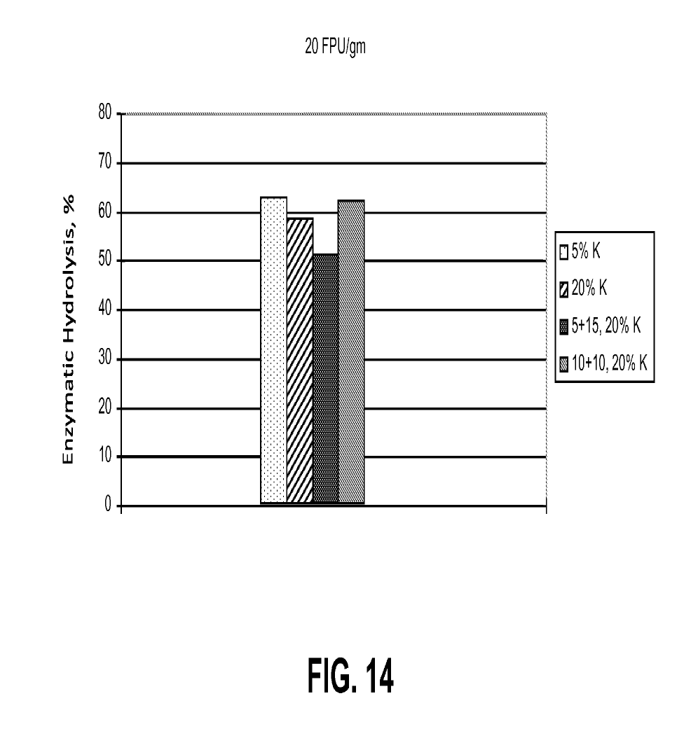

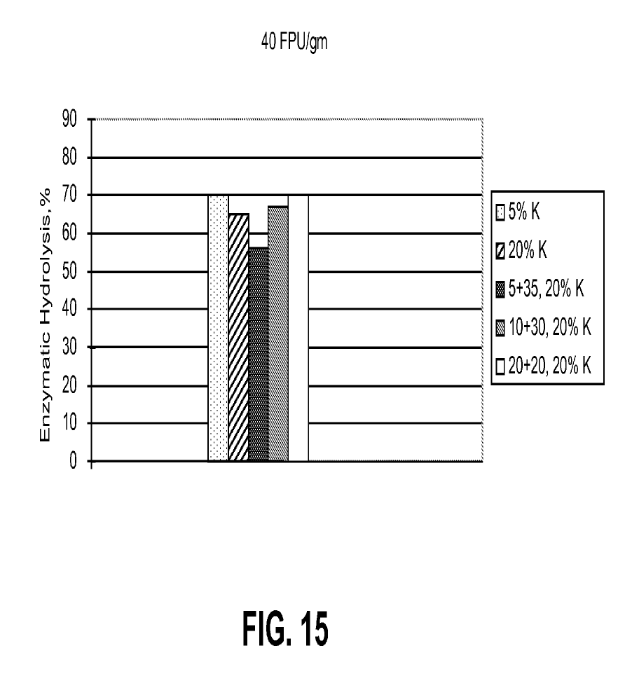

The effect of splitting the cellulase dose, so that some of the cellulase was added prior to thickening and some after thickening, was studied. Hardwood pulp that had been pretreated with green liquor as described in Example 1 (12% TTA) and having 28% lignin content was added to a dilution/mixing tank and diluted with water to 5% K. One portion (one eighth, one fourth, or one half) of the cellulase from a 10, 20, or 40 FPU/gram pulp enzyme dose was added, and the mixture was incubated for 10 minutes. After 10 minutes, the mixture was vacuum filtered to provide a thickened mixture having a fiber consistency of 20% K. The thickened mixture was allowed to hydrolyze for two hours and then the remaining enzymes (the second half of the cellulase and the xylanase and β-glucosidase) were added. The final mixture was allowed to hydrolyze for an additional 46 hours, filtered and the filtrate analyzed for monomeric sugar content. FIG. 13 shows how splitting the cellulase from a 10 FPU/gm pulp dose into two equal parts affected enzymatic hydrolysis. In addition to showing the results from a control sample where all of the cellulase was added prior to thickening, results from a sample hydrolyzed for 48 hours at 5% K are shown. FIG. 14 shows how splitting the cellulase charge so that only one fourth or one half of the total cellulase from a 20 FPU/gram pulp dose was added prior to thickening affected hydrolysis. Results are also provided from a sample where all the cellulase was added prior to thickening and from a sample where the mixture was not thickened. FIG. 15 shows how splitting the cellulase charge so that only one eighth, one fourth, or one half of the cellulase from a 40 FPU/gram pulp dose was added prior to thickening affected hydrolysis. Results are also provided for a sample where all of the cellulase was added prior to thickening to 20% K and a sample where hydrolysis was performed at 5% K.

Generally, the highest hydrolysis was observed when 50% of the cellulase charge was added to the pulp at low consistency and the remaining cellulase and other enzymes were added after thickening. Without being bound to any one theory, it appears that the initial cellulase charge can be well mixed with the pulp at low consistency and then serves to reduce viscosity in the thickened mixture so that the remaining enzymes can be easily mixed to contact and hydrolyze the remaining poly- and oligosaccharides.

Split enzyme addition at higher enzyme dosages was also explored. 82 FPU/gm cellulase was added to wood pulp at 5% K, mixed and thickened to 30% K. Based on the cellulase in the filtrate, it appeared that only about 10 FPU/gm of the cellulase remained in the thickened mixture. An additional 10 FPU/gm cellulase was added to the thickened mixture and the mixture was allowed to hydrolyze for 24 hours. A second 10 FPU/gm dose of additional cellulase was added and the mixture allowed to hydrolyze a further 24 hours. The mixture was then filtered and the remaining solids weighed. Based on weight loss compared to the biomass prior to hydrolysis, enzymatic efficiency was about 84%.

To a fresh pulp mixture at 5% K, 50 FPU/gm pulp of cellulase was added and mixed. The mixture was thickened via filtration to 30% K. Approximately 88% of the cellulase was recovered in the filtrate. An additional 6 FPU/gm pulp of cellulase was added to the 30% K mixture and it was allowed to hydrolyze for 24 hours. A further 6 FPU/gm pulp dose of cellulase was added and the mixture allowed to hydrolyze for a second 24 hours. The mixture was then filtered and the remaining solids weighed. Based on weight loss compared to the biomass prior to hydrolysis, enzymatic efficiency was about 78%.

### Example 7

### Filtrate Recycling

The recycling of filtrate from thickened enzymatic hydrolysis mixtures was studied to determine the effects on sugar yields. Recycling filtrate, for example, can make enzymatic hydrolysis process more economically efficient because enzymes in the filtrate can be reused.

Hardwood pulp was hydrolyzed at 5% K (20 L total volume) using 20 FPU/gm pulp enzymes (cellulase, xylanase and β-glucosidase) for 48 hours. Baseline sugar conversion was determined to be about 63-65%. The pulp was then thickened to a consistency of 20% K via vacuum filtration. The filtrate (15 L) was used as a part of the diluent for a new batch of pulp, which was diluted to 5% K overall and to which was also added a new 20 FPU/gram dose of enzymes. The new batch was allowed to hydrolyze for 48 hours. Sugar conversion of the new batch was determined to be about 70%. The new batch was then thickened to a consistency of 20% K via vacuum filtration. The filtrate (15 L) was used as part of the diluent for a second new batch of pulp. The second new batch of pulp was diluted to 5% K, mixed with a new 20 FPU/gram dose of enzymes, and hydrolyzed for 48 hours. Sugar conversion of the second new batch of pulp was analyzed and determined to be about 72%. Accordingly, it appears that recycling of the filtrate can be employed for increasing sugar concentration, leading to slightly higher overall pulp-to-sugar conversion.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of producing an alcohol from a lignocellulosic biomass, the method comprising:
providing lignocellulosic biomass;
contacting the lignocellulosic biomass with a first enzyme composition for a first period of time to provide a first hydrolysis mixture;
thickening the first hydrolysis mixture to form a second hydrolysis mixture;
hydrolyzing the second hydrolysis mixture for a second period of time to provide a fermentable sugar mixture; and
fermenting the fermentable sugar mixture to provide an alcohol.

2. The method of claim 1, wherein the lignocellulosic biomass is selected from the group consisting of herbaceous material, agricultural residues, forestry residues, municipal solid wastes, waste paper, pulp and paper mill residues, or a combination thereof.

3. The method of claim 2, wherein the lignocellulosic biomass is selected from the group consisting of corn stover, straw, bagasse, miscanthus, sorghum residue, switch grass, bamboo, water hyacinth, hardwood, hardwood chips, softwood chips, hardwood pulp, and softwood pulp.

4. The method of claim 1, further comprising pretreating the lignocellulosic biomass to increase enzymatic digestability.

5. The method of claim 4, wherein the pretreating comprises one or more of the group consisting of removing or altering lignin, removing hemicellulose, decrystallizing cellulose, removing acetyl groups from hemicellulose, reducing the degree of polymerization of cellulose, increasing the pore volume of lignocellulose biomass, and increasing the surface area of lignocellulose.

6. The method of claim 4, wherein the pretreating comprises one or more pretreatment technique selected from the group consisting of autohydrolysis, steam explosion, grinding, chopping, ball milling, compression milling, radiation, flow-through liquid hot water treatment, dilute acid treatment, concentrated acid treatment, peracetic acid treatment, supercritial carbon dioxide treatment, alkali treatment, organic solvent treatment, cellulose solvent treatment, and treatment with an aerobic fungi.

7. The method of claim 6, wherein the alkali treatment is selected from the group consisting of sodium hydroxide treatment, lime treatment, wet oxidation, ammonia treatment, and oxidative alkali treatment.

8. The method of claim 6, wherein the alkali treatment is green liquor treatment.

9. The method of claim 1, wherein contacting the lignocellulosic biomass with the first enzyme composition comprises mixing the lignocellulosic biomass with the first enzyme composition at a solids concentration of about 5%.

10. The method of claim 1, wherein the first hydrolysis mixture comprises between about 5 filter paper units (FPU) and about 85 FPU of lignocellulose-hydrolyzing enzyme per gram of lignocellulosic biomass.

11. The method of claim 10, wherein the first hydrolysis mixture comprises about 10 FPU of lignocellulose-hydrolyzing enzyme per gram of lignocellulosic biomass.

12. The method of claim 1, wherein the first enzyme composition comprises cellulase.

13. The method of claim 12, wherein the first enzyme composition further comprises xylanase and β-glucosidase.

14. The method of claim 12, wherein the first period of time ranges from about 1 minute to about 20 minutes.

15. The method of claim 14, wherein the first period of time ranges from about 5 minutes to about 10 minutes.

16. The method of claim 12, wherein contacting the lignocellulosic biomass with the first enzyme composition is performed at a temperature of between about 4° C. and about 70° C.

17. The method of claim 16, wherein the contacting is performed at a temperature of about 38° C.

18. The method of claim 12, wherein the contacting is performed at a pH of about 4.8.

19. The method of claim 1, wherein the thickening step comprises increasing the fiber concentration of the first hydrolysis mixture to provide a second hydrolysis mixture having a solids concentration of between about 15% and about 30%.

20. The method of claim 19, wherein the thickening step comprises filtering the first hydrolysis mixture to provide the second hydrolysis mixture and a filtrate.

21. The method of claim 20, wherein the filtering is performed by vacuum filtering the first hydrolysis mixture using a filter press.

22. The method of claim 20, wherein the filtrate comprises about 80% of the liquid from the first hydrolysis mixture.

23. The method of claim 20, wherein the filtrate comprises water and unabsorbed lignocellulose-hydrolyzing enzyme, and wherein said filtrate is used to dilute lignocellulosic biomass, thereby recycling the lignocellulose-hydrolyzing enzyme.

24. The method of claim 23, wherein the first enzyme composition comprises cellulase and wherein the filtrate comprises about 10% to about 20% of the cellulase from the first hydrolysis mixture.

25. The method of claim 1, wherein the second period of time ranges between about 1 day and about 3 days.

26. The method of claim 25, wherein hydrolyzing the second hydrolysis mixture comprises hydrolyzing the second hydrolysis mixture for a first portion of the second period of time, adding a second enzyme composition to the second hydrolysis mixture to increase the enzyme dosage in the second hydrolysis mixture, and continuing hydrolysis of the second hydrolysis mixture for a second portion of the second period of time to provide the fermentable sugar mixture.

27. The method of claim 26, wherein the first portion of the second period of time ranges between about 0 hours and about 24 hours.

28. The method of claim 27, wherein the first portion of the second period of time ranges between about 2 hours and about 3 hours.

29. The method of claim 26, wherein the second enzyme composition comprises xylanase and β-glucosidase.

30. The method of claim 29, wherein the second enzyme composition further comprises cellulase.

31. The method of claim 30, wherein the first enzyme composition and the second enzyme composition each comprise cellulase, and the first enzyme composition comprises between about 25% and about 50% of the total cellulase dosage from the first and second enzyme compositions.

32. The method of claim 31, wherein the first enzyme composition comprises about 50% of the total cellulase dosage.

33. The method of claim 26, wherein the second portion of the first period of time ranges between about 24 hours and about 48 hours.

34. The method of claim 1, wherein hydrolysis efficiency of cellulosic material originally present in the lignocellulosic biomass is 70% or greater.

35. The method of claim 34, wherein the hydrolysis efficiency is between about 78% and about 84%.

36. The method of claim 1, wherein the fermentable sugar mixture comprises about 12% fermentable sugar by volume.

37. The method of claim 1, wherein fermenting comprises fermenting the fermentable sugar mixture using a microorganism to provide an alcohol mixture and distilling the alcohol mixture to provide the alcohol.

38. The method of claim 37, wherein the microorganism is yeast.

39. The method of claim 37, wherein the alcohol mixture comprises about 6% alcohol by volume.

40. The method of claim 37, further comprising dehydrating the alcohol.

41. The method of claim 1, wherein the alcohol is ethanol.

42. A composition comprising an alcohol prepared according to the method of claim 1.

43. The composition of claim 42, wherein the alcohol is ethanol.

44. The composition of claim 43, wherein the composition comprises 95% or greater ethanol by volume.

45. The composition of claim 43, wherein the composition is a fuel mixture comprising ethanol and gasoline.
